(19) United States
(12) Patent Application Publication
(10) Pub. No.: US 2026/0168996 A1
(43) Pub. Date: Jun. 18, 2026

(54) TITRATION METHOD FOR MEASURING KINETIC BINDING PARAMETERS AND FOR DISCRIMINATING SPECIFIC BINDING FROM BACKGROUND

(71) Applicant: Miltenyi Biotec B.V. & Co KG, Bergisch Gladbach (DE)

(72) Inventor: Ali KINKHABWALA, Bergisch Gladbach (DE)

(73) Assignee: Miltenyi Biotec B.V. & Co KG, Bergisch Gladbach (DE)

(21) Appl. No.: 19/123,704

(22) PCT Filed: Nov. 1, 2023
(86) PCT No.: PCT/EP2023/080498

§ 371 Date: Apr. 23, 2025

(30) Foreign Application Priority Data
Nov. 2, 2022 (EP) 22205099.9

(51) Int. Cl. G01N 33/557

(52) U.S. Cl. CPC G01N 33/557

(57) ABSTRACT

Method for determination of the kinetic binding parameters for a molecule binder conjugated to a fluorophore. Repeated staining of the antigen and detection of the emission radiation generates a titration series. Fitting of the emission radiation over the titration series allows for determination of kinetic binding parameters. If the emission radiation is detected with a camera, single pixel fitting using a global analysis permits discrimination of the specific binding signal from non-specific background in each pixel.

The invention is directed to a method for measuring kinetic binding parameters for a molecule binder with its target epitope and for discrimination of specific binding from background.

### BACKGROUND

The state-of-the-art approach for measuring kinetic binding parameters for a molecule binder with its target epitope is based on purification of the target epitope to isolate the interaction in vitro. Typically, binding of the molecule to its target epitope is monitored in a label-free way by immobilizing the target epitope on a glass substrate, with the on-rate constant and off-rate constant of the molecule binder measured via changes in surface plasmon resonance (SPR) at the glass substrate (e.g. Bakhtiar, J Chem Ed 90, 203, 2012). Specifically, empty buffer is applied to the sample and then rapidly exchanged with buffer containing the molecule binder at a particular concentration to determine the on-rate constant, kₒₙ, defined according to mass action as the proportionality constant connecting the rate of change of the concentration, B, of epitopes bound with the molecular binder to the product of the unbound molecule binder concentration, A, and the unbound target epitope concentration, E:

$$\frac{dB}{dt} = {k}_{on} AE .$$

The sample is then typically allowed to reach equilibrium binding to determine the dissociation constant. The buffer is then rapidly exchanged with empty buffer, allowing independent determination of the off-rate constant, defined as the proportionality constant connecting the rate of change in the concentration of bound epitopes with the concentration of bound epitopes:

$$\frac{dB}{dt} = - {k}_{off} B .$$

While such in vitro approaches are routinely employed for determination of the on-rate constant and off-rate constant of molecule binders, purification of the target epitope represents a complicated and time-consuming step. Purification may also not even be possible for certain epitopes. Attachment of the target epitope to glass represents an additional problematic step, as attachment can lead to misfolding and altered binding. The molecule binder may also additionally and non-specifically bind the glass substrate, creating a background. Finally, the kinetic parameters obtained by such in vitro approaches may not be transferable to other more relevant contexts, e.g. to accurately predict the binding of a dye-labeled version of the molecule binder to a fixed biological sample for immunofluorescence.

In situ determination of the kinetic binding constants of a fluorescent probe binding to epitope targets within a biological sample has been reported e.g. at the level of cell populations (Bondza et al., Frontiers in Immunology 8, 455, 2017). Here, a LigandTracer Green setup (Ridgeview Instruments) was employed to acquire the kinetic binding curve for an immunofluorescent probe applied to a cell population. Specifically, the target cell population was briefly stained (30 sec), washed (5 sec), and imaged (30 sec) with a microscope in multiple cycles. The kinetic binding curve of the cell population could thereby be sampled at the roughly 1 min timescale at a fixed concentration of applied probe. Following incubation with the probe at this fixed concentration, further incubation steps at zero probe concentration could be used to determine the off-rate constant. Background binding was corrected for by obtaining a binding curve for control cells that did not express the target epitope, with this control binding curve subtracted from the binding curve of the target cell population. Related approaches down to the level of single cells (May et al., Molecular Pharmacology 78, 511, 2010) or tissue regions (Dubois et al., BMC Research Notes 6, 542, 2013) have been reported that also relied on separate measurements of control cells or regions to indirectly estimate the contribution of background binding in the targeted cells or regions. M ore precise determination of the actual contributions of specific vs. non-specific binding within each region of interest and, ideally, down to the single pixel level was not achieved.

Immunofluorescence staining of cells has recently been extended from classical single-shot imaging to the imaging of multiple different targets on the same tissue slice, allowing for high-content insight into target epitopes and the cellular microenvironments of a tissue (Kinkhabwala et al., Sci Rep 12, 1911, 2022). The MACSima Imaging System (Miltenyi Biotec B.V. & Co. KG) is especially designed for this technology.

The state-of-the-art solution to reduce the background in immunofluorescence images is through “blocking” the sample before staining using specific blocking reagents (e.g. Fc domain to block Fc receptors) or non-specific blocking reagents (e.g. full serum, bovine serum albumin, or isotype antibody control). The amount of blocking (concentration of blocking reagent and incubation time) required to enhance the signal-to-background for a given antibody staining is hard to predict. While blocking can reduce the background and thereby improve the contrast of an antibody stain, it does not remove the background entirely. Blocking reagents can also block the targeted epitopes in the sample, reducing the specific signal.

The background pattern in the image can be visualized through use of an isotype antibody control staining using a different fluorescent label. Scaled subtraction of the isotype control staining from the immunofluorescence staining can be used to remove the background signal. However, the scale factor to apply is not well-defined. Chromatic shifts or other imaging distortions created by detection across two different fluorescence channels may also corrupt the final image. The isotype control staining may also not represent the actual non-specific binding of the antibody due to the necessary differences in the recognition domain.

There is no currently available and sufficiently general method for the accurate determination of the kinetic binding parameters (specifically, the on-rate constant and off-rate constant) governing the binding of a molecule binder in situ to target epitopes within a fixed biological sample. The fundamental challenge here is proper discrimination of the specific signal (targeted binding interaction) from multiple potential contaminating backgrounds (largely due to non-specific binding interactions).

There is also no currently available and sufficiently general method for reliable extraction of the specific staining signal from background in each pixel of an immunofluorescence image of a fixed biological sample, which would allow for truly background-free immunofluorescence imaging.

### OBJECT OF THE INVENTION

We propose a method (“titration method”) based on the iterative staining and imaging of a dye-labeled molecule binder to a fixed biological sample using an automated microscope for immunofluorescence imaging that (1) allows for accurate discrimination of the specific kinetics of binding of the molecule to its target epitope from background binding interactions, and (2) simultaneously allows for accurate discrimination of specific binding signal from background in every pixel of an image of the sample. Specifically, a model is fit across a stack of images individually corresponding to different titrations of the dye-labeled molecule binder to the sample. Fitting of a model describing both specific and background binding interactions allows for simultaneous extraction of the kinetic binding parameters characterizing the specific interaction, as well as the fraction of signal to background in every pixel (which represents a 3D voxel) of the sample image. The main outputs of our method are therefore the kinetic binding parameters for the molecule binding its specific target epitope, as well as a pure signal image and a pure background image.

Object of the invention is therefore a method for determining the on-rate constant for specific binding of a conjugate comprising a fluorescent detection moiety and an antigen binding moiety applied to a fixed biological sample expressing the corresponding antigen, as well as for determining the contribution to the emission radiation of specific binding and background binding in each pixel of the image of the fixed biological sample, characterized by the steps
a. measuring the emission radiation of the fixed biological sample as an image formed on a camera before providing the conjugate
b. providing the conjugate to the fixed biological sample subsequently in at least two different concentrations and for a specified time interval
c. detecting the emission radiation for each concentration as an image formed on a camera
d. aligning the images to each other
e. fitting a function that accounts for the amount of specific binding and background binding for each concentration to the emission radiation within each aligned pixel over the separate images
f. obtaining from step e) the on-rate constant that specifies the specific binding function

Preferable, in addition to obtaining the on-rate that specifies the specific binding function, the contribution to the emission radiation of specific binding and background binding in each aligned pixel of the image of the fixed biological sample is determined.

The method can be further characterized by
g. creating an image of specific binding by assigning the emission radiation contributed by specific binding to each aligned pixel
h. creating an image of background binding by assigning the emission radiation contributed by background binding to each aligned pixel.

The fixed biological sample could correspond to one of the following: adherent cells, suspension cells, tissue, or smears (e.g. bone marrow).

### BRIEF DESCRIPTION OF THE DRAWINGS

FIGS. 1A and B illustrate the titration method of the invention

FIG. 2 shows an example of the titration method applied to a fixed tissue slice

### DESCRIPTION OF THE INVENTION

Detailed here is a method for measuring the kinetic binding parameters for a molecule binder to its target epitope located in a fixed biological sample. The method is based on the application of a series of titrations of the molecule binder to the fixed biological sample, with the emission radiation from the sample measured after each step. If the emission radiation is detected as a microscope image for each titration step, a global analysis over the aligned images (corresponding to the different titrations) can be used. Global analysis, in this case, allows for discrimination of the signal (specific binding of the molecule binder) from the background (unspecific binding of the molecule binder) in each pixel of the aligned image series. A new image can therefore be constructed that contains only the signal in each pixel, which is proportional to the concentration of the target epitope.

In the drawings the following reference numbers are used to refer to the following features. Similar reference numbers are used in the various figures to refer to components that serve a similar or identical function.
001 Fixed biological sample
002 Coverslip glass
003 Microscope objective
004 Image of nuclear DA PI staining
005 Image of dye-labeled molecule binder (contrast the same as other images in the row)
006 Image of dye-labeled molecule binder (arbitrary contrast)
007 Extracted image of specific binding (“Signal”)
008 Extracted image of unspecific binding (“Background”)

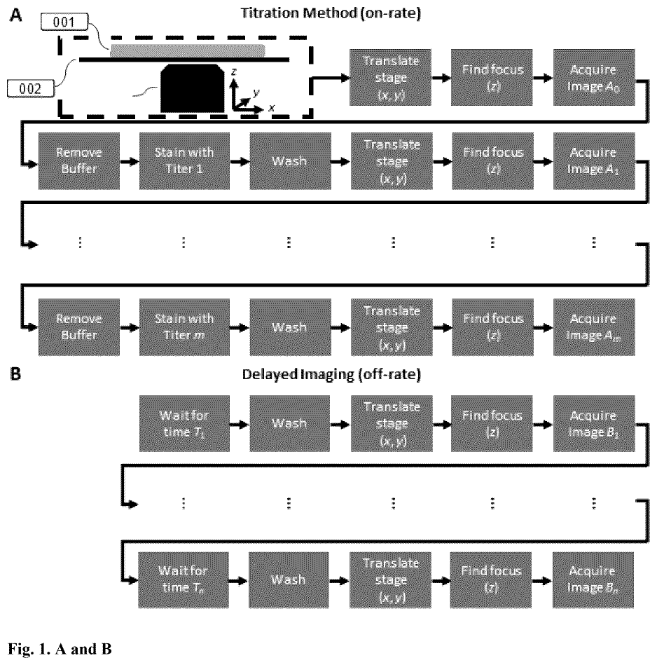

The titration method is depicted in FIG. 1A. The titration method is characterized by a series of stainings and washings of a single molecule binder that targets specific antigens within a fixed biological sample, typically corresponding to a fixed, few-micron-thick tissue slice. The resultant series of images, Aₘ, for the different titration steps, m, can be used, for example, to determine the on-rate constant for specific binding of a molecule binder. The image series can also be used to discriminate the contribution of signal from background binding. In FIG. 1B, an additional delayed series of images comprised of at least one image, B₁, can be carried out immediately following the titration process depicted in FIG. 1A for better determination and discrimination of the off-rate constants for specific binding and for background binding.

With a modification of the standard instrument protocol, the titration method of the invention can be performed with the M A CSima Imaging System (Miltenyi Biotec B.V. & Co. KG), which allows for automated, serial immunofluorescence staining of a fixed biological sample.

The standard instrument protocol for the M A CSima consists of serial staining and immunofluorescence imaging of a fixed biological sample with an array of molecule binders. The standard protocol is normally characterized by iterative cycles of staining, washing, imaging, and erasure. Erasure of the fluorescence signal from a particular molecule binder is accomplished by photodestruction of the fluorophore (photobleaching) or by enzymatic cleavage of the molecule binder to remove the fluorophore from the sample (with an additional washing step applied to remove the solubilized fluorophore). For the serial staining of a given field of view, return to the same z-position in the sample is assured through detection and adjustment of the distance of the objective to the glass coverslip (with position of the glass determined by detection of reflected IR light) and/or through comparison of the current DA PI image of the sample with the initial DA PI image from the first cycle. Alignment of the images in the xy-plane to sub-pixel precision is then performed during a later image registration step, allowing for precise measurement of the same voxels of the fixed biological sample across the final aligned image stack.

For implementation of the titration method on the M A CSima, the following slight changes to the standard instrument protocol are required. The titration method is based on iterative cycles of repeated staining (typically for 10 min), washing, and imaging for a single molecule binder, with erasure no longer applied in each cycle. Instead, a series of concentrations is applied to the sample in an additive fashion. For example, serial application of 0.625 μg/mL, 1.875 μg/mL, 7.5 μg/mL, and 30 μg/mL of the molecule binder would amount to a four-fold additive staining increase at each titration step of 0.625 μg/mL, 2.5 μg/mL, 10 μg/mL, and 40 μg/mL. The range of concentrations should be carefully chosen to ensure sufficient sampling of the full shape of the as-yet-unknown saturation curve. Here, it is critical that the highest concentration(s) bring the molecule binder into at least slight saturation, which is similar to the standard requirement for measuring the dissociation constant in the context of a chemical binding assay.

Weak, non-specific interactions of the molecule with the sample should not exhibit saturation over the applied titration range and would be expected to increase linearly with the additive concentration. However, more complicated background models can also be accommodated (see the Mathematical Treatment section below).

Such background models can be utilized in a first embodiment of the invention to further obtain the off-rate constant for specific binding and the off-rate constant for background binding from the function.

In a second embodiment, the on-rate constant for background binding is further obtained from the function.

The different profiles expected for a saturating signal vs. a linearly increasing background (or more complicated background model) over the series of titration images therefore represent the key aspect of the titration method that allows for reliable discrimination of signal from background.

Specifically, fitting of the integrated signal (or global fitting based on individual pixel information) for each individual image of the titration series allows for precise determination of the specific on-rate constant of the molecule with its targeted epitopes.

Further, one or more images can be acquired at fixed timepoints following the final staining/washing step to allow for separate and more direct determination of the off-rate constant for specific binding and the off-rate constant for background binding (FIG. 1B).

Accordingly, in a third embodiment, before step d, the following steps are performed
j. waiting a specific time interval
k. detecting the emission radiation.

Steps j and k can be repeated at least one time with the same or a different time interval.

The method can be further characterized by replacing step f with
l. obtaining from e the on-rate constant and the off-rate constant that specifies the specific binding function.

The method can be further characterized by fitting the function in e using a global analysis.

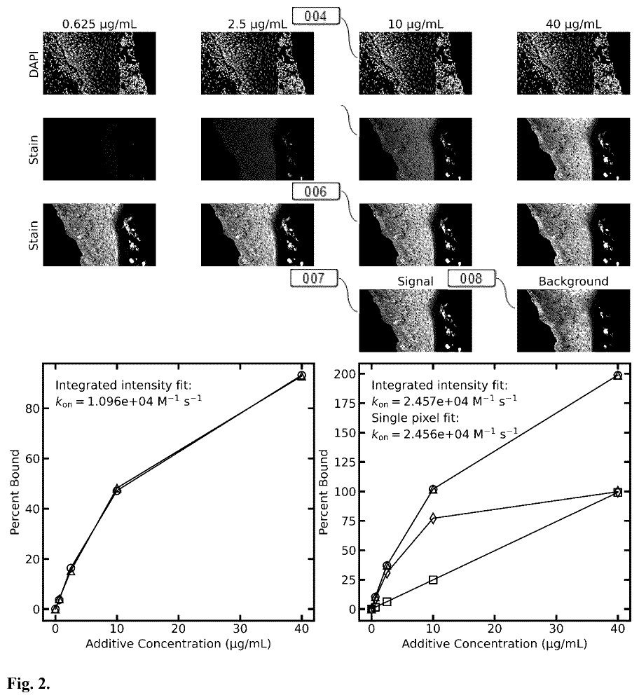

A global analysis of the titration image series can be used to determine both the optimal global kinetic parameters for the specific binding (and, if necessary, the kinetic parameters for the background binding) as well as the local parameters corresponding to the fractional contribution made by the specific binding vs. background binding in each pixel. The latter allows for reconstruction of images containing only specific binding or only background binding (“Signal” and “Background” images in FIG. 2). The image of specific binding is importantly free from contamination by background binding in each pixel to the noise limit, allowing for “background-free” immunofluorescence imaging.

The method can be further characterized by fitting the function in e in two steps
m. in a first step, fitting to the integrated image intensities to determine the specific binding function
n. in a second step, fitting for the amount of specific binding and background binding for each concentration to the emission radiation within each aligned pixel over the separate images.

In the following, all embodiments are described in detail.

### Mathematical Treatment

In the above, the titration process, analysis, and outputs corresponding to the various embodiments were described only schematically. In the following, a precise mathematical treatment is given. We assume throughout the following parameters:
V volume of solution
A_{T} total applied antibody
A free antibody
α_{T} total applied antibody concentration (A_{T}/V)
B bound antibody
E_{T} total epitope
E free epitope
b fraction of bound epitope (B/E_{T})
f fraction of free epitope (E/E_{T})
ε ratio of total epitope to total antibody (E_{T}/A_{T})
kₒₙ On-rate constant [μM₋₁ s₋₁]
K_{off} Off-rate constant [s⁻¹]
K_{D} Dissociation constant [μM]
   For the case of a standard immunofluorescence staining, a fixed biological sample is attached to coverslip glass at the bottom of a well and then immersed with a total amount of antibody probe, A_{T}, in solution volume, V. The total number of epitopes in the sample is E_{T}. Conservation conditions mandate:

$$\begin{matrix}{A}_{T} = A + B \\ {E}_{T} = E + B\end{matrix}$$

where B is the bound antibody (in a 1:1 binding to the target epitope), A is the free antibody, and E is the remaining unbound epitope.

Assumption #1 (fast diffusion, no significant spatial gradients): If diffusion is fast compared to the binding timescale, then the exact spatial distribution of the epitopes in the staining volume can be neglected and we can simply examine the increase of total bound epitope over time based on standard chemical kinetics defined by the kₒₙ and k_{off} for the interaction:

$$\frac{d}{dt} ( \frac{B}{V} ) = {k}_{on} \frac{A}{V} \frac{E}{V} - {k}_{off} \frac{B}{V}$$

$$\frac{dB}{dt} = \frac{{k}_{on}}{V} AE - {k}_{off} B$$

$$\frac{dB}{dt} = \frac{{k}_{on}}{V} ( {A}_{T} - B ) ( {E}_{T} - B ) - {k}_{off} B$$

Defining the binding fraction as b=B/E_{T} gives:

$$\frac{db}{dt} = {k}_{on} {a}_{T} ( 1 - ε b ) ( 1 - b ) - {k}_{off} b$$

with the total applied antibody concentration α_{T}=A_{T}/V and a total epitope-to-antibody ratio of ε=E_{T}/A_{T}. While the above equation could theoretically be solved for the temporal evolution of the bound fraction, further assumptions relevant for standard immunofluorescence staining are detailed immediately below that lead to even simpler analytical results.

Assumption #2 (antibody greatly exceeds targeted epitopes): For a typical immunofluorescence staining, the amount of applied antibody is much greater than the total number of targeted epitopes in the sample: E_{T}<<A_{T}. In this ε→0 limit, we obtain:

$$\frac{db}{dt} = {k}_{on} {a}_{T} ( 1 - b ) - {k}_{off} b$$

The general solution to the above differential equation is (Equation 1):

$$b ( t ) = \frac{1}{1 + \frac{{k}_{off}}{{k}_{o n} {a}_{T}}} [ 1 - ( 1 - ( 1 + \frac{{k}_{off}}{{k}_{o n} {a}_{T}} ) {b}_{0} ) exp ( - {k}_{o n} {a}_{T} ( 1 + \frac{{k}_{off}}{{k}_{o n} {a}_{T}} ) t ) ]$$

Taking b₀=0 gives (Equation 2):

$$b ( t ) = \frac{1}{1 + \frac{{k}_{off}}{{k}_{o n} {a}_{T}}} [ 1 - exp ( - {k}_{o n} {a}_{T} ( 1 + \frac{{k}_{off}}{{k}_{o n} {a}_{T}} ) t ) ]$$

### Determination of the Specific Interaction kₒₙ

Assumption #3a: If the off-rate constant is negligible (i.e. k_{off}<<kₒₙα_{T} or, equivalently, α_{T}>>K_{D}), Equation 2 becomes:

$$b ( t ) = 1 - exp ( - {k}_{o n} {a}_{T} t )$$

Assumption #3b: Alternatively, if the argument in the exponent in Equation 2 is small compared to 1 (far from saturation), then first-order Taylor expansion gives the following linear relationship:

$$b ( t ) = {k}_{o n} {a}_{T} t$$

Under either assumption, the off-rate constant plays no role in the evolution of the bound fraction.

Following immunostaining with antibody concentration α_{T}=α₁ for some duration t=t₁, the sample is washed and imaged. Under Assumption 3a, the observed bound fraction is:

$${b}_{1} = 1 - exp ( - {k}_{o n} {a}_{1} {t}_{1} )$$

Iterative stainings are more easily expressed in terms of the exponential reduction of the free epitope (fᵢ=1−bᵢ):

$${f}_{1} = {f}_{0} exp ( - {k}_{o n} {a}_{1} t )$$

For multiple stainings (assuming f₀=1), this can be written as:

$${f}_{n} = {f}_{n - 1} exp ( - {k}_{o n} {a}_{n} {t}_{n} ) = ⋯ = {\prod }_{i = 1}^{n} exp ( - {k}_{o n} {a}_{i} {t}_{i} ) = exp ( - {k}_{o n} {\sum }_{i = 1}^{n} {a}_{i} {t}_{i} )$$

If the immunostaining incubation time is kept constant in each step (t=tₛ), then:

$${f}_{n} = {f}_{n - 1} exp ( - {k}_{o n} {a}_{n} {t}_{s} ) = ⋯ = {\prod }_{i = 1}^{n} exp ( - {k}_{o n} {a}_{i} {t}_{s} ) = exp ( - {k}_{o n} {t}_{s} {\sum }_{i = 1}^{n} {a}_{i} )$$

The iterative immunostaining at each step k is therefore equivalent to a single staining of duration t=tₛ for the “additive” concentration

$${α}_{k} = {\sum }_{i = 1}^{k} {a}_{i}$$

(with K=n for the final staining). The bound fraction at each step k is then simply:

$${b}_{k} = 1 - {f}_{k} = 1 - exp ( - {k}_{o n} {t}_{s} {\sum }_{i = 1}^{k} {a}_{i} ) = 1 - exp ( - {k}_{o n} {t}_{s} {α}_{k} )$$

### Determination of the Specific Interaction kₒₙ and k_{off}

More generally, if the off-rate constant cannot be neglected, the solutions are best expressed in matrix form in the following manner, with different incubation times, tₖ, allowed at each step. According to Equation 1, the bound fraction at step k (following application of the titer αₖ) in terms of the bound fraction at step k−1 is:

$${b}_{k} = \frac{1}{1 + \frac{{k}_{off}}{{k}_{o n} {a}_{k}}} ( 1 - exp ( - {k}_{o n} {a}_{k} ( 1 + \frac{{k}_{off}}{{k}_{o n} {a}_{k}} ) t ) ) + {b}_{k - 1} exp ( - {k}_{o n} {a}_{k} ( 1 + \frac{{k}_{off}}{{k}_{o n} {a}_{k}} ) t )$$

### Introducing,

$${β}_{k} = 1 + \frac{{k}_{off}}{{k}_{o n} {a}_{k}}$$

and γₖ=exp(−Kₒₙαₖβₖtₖ), these expressions become:

$${b}_{k} = \frac{1 - {γ}_{k}}{{β}_{k}} + {γ}_{k} {b}_{k - 1}$$

The above can be re-expressed in the following convenient matrix form:

$$( \begin{matrix}1 \\ {b}_{k}\end{matrix} ) = ( \begin{matrix}1 & 0 \\ \frac{1 - {γ}_{k}}{{β}_{k}} & {γ}_{k}\end{matrix} ) ( \begin{matrix}1 \\ {b}_{k - 1}\end{matrix} ) = {S}_{k} ( \begin{matrix}1 \\ {b}_{k - 1}\end{matrix} ) = {S}_{k} {S}_{k - 1} … {S}_{1} ( \begin{matrix}1 \\ 0\end{matrix} )$$

Here, an initial bound fraction of zero is assumed (b₀=0). Note that each Sₖ matrix depends on four parameters: Sₖ≡Sₖ(kₒₙ, k_{off}, αₖ, tₖ).

If there are long wait times between the separate incubations (for which the applied antibody concentration is zero), the off-rate constant might play a significant role. These intervals can be explicitly included in the model as well. Here, we assume negligible rebinding of antibodies that have fallen off of the epitopes, as any change in bound fraction due to the newly freed antibodies is considered negligible in comparison to the change in bound fraction occurring during the staining steps for which the applied antibody is assumed to be in great excess to the epitopes (Assumption #2, E_{T}<<A_{T} for the stainings). The bound epitope is therefore reduced by:

$${b}_{k} = exp ( - {k}_{off} {t}_{k} ) {b}_{k - 1}$$

Defining θₖ=exp(−k_{off}tₖ), this is just:

$${b}_{k} = {θ}_{k} {b}_{k - 1}$$

which in matrix form is:

$$( \begin{matrix}1 \\ {b}_{k}\end{matrix} ) = ( \begin{matrix}1 & 0 \\ 0 & {θ}_{k}\end{matrix} ) ( \begin{matrix}1 \\ {b}_{k - 1}\end{matrix} ) = {W}_{k} ( \begin{matrix}1 \\ {b}_{k - 1}\end{matrix} )$$

In case of a sequence of four iterative staining steps (S₁, S₃, S₅, S₇), alternated with three wait steps (W₂, W₄, W₆), the final bound fraction can be determined from:

$$( \begin{matrix}1 \\ {b}_{7}\end{matrix} ) = {S}_{7} {W}_{6} {S}_{5} {W}_{4} {S}_{3} {W}_{2} {S}_{1} ( \begin{matrix}1 \\ 0\end{matrix} )$$

While the final bound fraction, b₇, is algebraically complicated, it is only dependent on the up to two fitting parameters, kₒₙ and optionally k_{off}, as well as an overall normalization factor.

In the above, we have considered only the fraction of bound epitope within the total volume. In the case of an image, the relative intensity from one pixel to the next will depend on the local concentration of target epitope contributing to the staining of pixel p, which we will refer to as eₚ. Here, eₚ specifically refers to the convolution of the true 3D concentration of the epitope with the optical transfer function (“detection volume”) of the microscope (e.g. for a confocal microscope, the optical transfer function is well approximated by a spatially invariant 3D Gaussian with axes σₓ=σ_{y}, σ_{z} for each pixel). The intensity in the immunofluorescence image of the specific signal following titration step k is then simply:

$${S}_{p}^{k} = E {e}_{p} {b}_{k}$$

where E is a global normalization factor that will depend on the excitation intensity, exposure time, pixel quantum efficiency, etc.
Determining Specific Interaction (kₒₙ and/or k_{off}) and Linear Background (Slope, m)

In the case of significant contribution from background, the background is expected to increase linearly with the applied titration. It was shown above (Approximation #3b) that a linear approximation is appropriate for a binding interaction that is far from saturation, which is a reasonable assumption for the background and also agrees with our empirical results (e.g. FIG. 2). As the non-specific sites are heterogeneous, they will be expected to differ in interaction strength, with a different on-rate constant, con, required for each distinct class of non-specific binding site, j. A mixture of different non-specific sites is of course also possible within a single pixel of an immunofluorescence image. The bound fraction for the type j binding sites after incubation with applied concentration, α_{T}, for time, tₛ, will then be (according to Approximation #3b):

$$b ( t ) = {k}_{o n}^{j} {a}_{T} t$$

### If

$${c}_{p}^{J}$$

is the concentration of non-specific binding sites of type j that contribute to the intensity of pixel p, then the observed pixel intensity due to the total non-specific background after the first incubation step (of duration tₛ) will be:

$${D}_{p}^{1} = N {\sum }_{j} {c}_{p}^{j} {k}_{o n}^{j} {t}_{s} {a}_{1}$$

$${D}_{p}^{1} = N ( {\sum }_{j} {c}_{p}^{j} {k}_{o n}^{j} ) {t}_{s} {a}_{1}$$

$$or , simply ,$$

$${D}_{p}^{1} = N {m}_{p} {a}_{1}$$

where N is the same normalization constant as above for the specific binding and mₚ is a linear slope for the background specific for pixel p and defined as the sum of the products of the non-specific binding site concentrations and on-rate constants together with the incubation time tₛ. Note that, in the linear approximation, it is unnecessary to specify the underlying concentrations or on-rate constants across the different classes of non-specific binding sites that contribute to the single pixel parameter mₚ.

If the titrations are performed in sufficiently rapid succession, then the off-rate constant for non-specific binding can be neglected, implying a linear increase of background upon each titer step, which is dependent only on the “additive” concentration,

$${α}_{k} = {\sum }_{i = 1}^{k} {a}_{i}$$

(assuming equal incubation times for each step of tₛ):

$${D}_{p}^{k} = E ( {\sum }_{j} {c}_{p}^{j} {k}_{o n}^{j} ) {t}_{s} {\sum }_{i = 1}^{k} {a}_{i}$$

$${D}_{p}^{k} = E {m}_{p} {α}_{k}$$

The model-predicted intensity in pixel p at step k is then the sum of the specific signal and non-specific background contributions with an additional offset, Qₚ, included that is assumed independent of the step k (e.g. to account for imperfect subtraction of the pre-stain image intensity from all subsequent staining images):

$${H}_{p}^{k} = {S}_{p}^{k} + {D}_{p}^{k} + {Q}_{p} = E ( {e}_{p} {b}_{k} + {m}_{p} {α}_{k} ) + {Q}_{p}$$

The specific binding fraction at step k is calculated as explained above (matrix form). Defining Nₚ=Eeₚ and Mₚ=Emₚ, we obtain for the above:

$${H}_{p}^{k} = {N}_{p} {b}_{k} + {M}_{p} {α}_{k} + {Q}_{p}$$

Here, up to three local parameters are required for single pixel fitting across the titer image series: Nₚ, Mₚ, and optional Qₚ. These are fit along with the global values for the kₒₙ and optional k_{off} for the specific interaction. We can typically neglect k_{off} over the duration of the titration process, which gives:

$${H}_{p}^{k} = {N}_{p} ( 1 - {e}^{- {k}_{o n} {t}_{s} {α}_{k}} ) + {M}_{p} {α}_{k} + {Q}_{p}$$

A more complicated model for the background than the above simple linear model could also be considered (e.g. similar in form to the specific staining model component but specified by a different kₒₙ).

To perform a properly weighted fit of the model to the pixel information across the image series, an error model is required. A Gaussian model, op, provides a very good approximation for the uncertainty of the observed intensity in each pixel of each image, accounting well for the Poisson shot noise arising from photon counting statistics and also for the typically Gaussian camera readout noise (important at low intensities). Another significant contribution to the uncertainty may come from the binding site occupation statistics, which are drawn from an underlying binomial distribution, but can also be approximated as a Gaussian uncertainty. In the case that no noise model is available, a simple Gaussian approximation for the Poisson shot noise distribution (or the binomial distribution for binding site occupation) can be made:

$${σ}_{p}^{k} ∝ \sqrt{{I}_{p}^{k}} ,$$

with the proportionality factor (determined in part by the camera gain) assumed to be the same across all pixels of the camera. In this case, the exact value of the proportionality factor is not required to perform the fitting.

Now that the error model is defined, we can now perform a properly weighted global fitting of the modeled intensities to the observed intensities. Specifically, this entails minimization of the squared differences of the modeled and observed intensities across all pixels p of each titer image k for a given set of local (Nₚ, Mₚ, Qₚ) and global (kₒₙ and k_{off}) model parameters:

$$C = {\sum }_{k} {\sum }_{p} {\frac{1}{{( {σ}_{p}^{k} )}^{2}} [ {H}_{p}^{k} - {I}_{p}^{k} ]}^{2}$$

Here, we assume that the images have been acquired at the same axial plane and have been laterally registered to each other after acquisition. Return to the same axial plane can be achieved by precisely measuring the distance of the objective to the coverslip glass or by reference to a control image obtained in a different channel (e.g. using a transmission image or a nuclear staining using DA PI). Approximate return to the same lateral position can be insured by using an accurate stage to return to the same region of interest for each imaging step. Alternatively, the sample could be held at a static position through the entirety of the titration process. The lateral accuracy of the instrument positioning need not be as accurate as the axial accuracy, as the different titer images can be laterally aligned to sub-pixel precision after their acquisition using standard image registration algorithms. Proper registration in all three spatial dimensions ensures that, for a given pixel, the exact same voxel is addressed in each aligned image of the titration series.

Global fitting can be performed at the full pixel resolution of the images, or for any arbitrary partitioning of the data, e.g. into super-pixels of size 2×2, 3×3, etc. For the latter, we now need to simply sum over each super-pixel with index s instead of p in the above definition of the least-squares sum:

$$C = {\sum }_{k} {\sum }_{s} {\frac{1}{{( {σ}_{s}^{k} )}^{2}} [ {H}_{s}^{k} - {I}_{s}^{k} ]}^{2}$$

The maximum number of independent data points over a series of K total images, each with P pixels, is A=K×P. If the images are rebinned into a total number of super-pixels, S<P, then 4=K×S.

The most extreme partitioning possible would amount to a single super-pixel (S=1) corresponding to the full image itself, with the total image intensity, I^{k}, corresponding to a simple sum over all the pixel intensities. In this particular case, the least-squares sum becomes:

$$C = {\sum }_{k} {\frac{1}{{( {σ}^{k} )}^{2}} [ {H}^{k} - {I}^{k} ]}^{2}$$

with the Gaussian uncertainty of the integrated image intensity calculated in the standard way as

$${( {σ}^{k} )}^{2} = {\sum }_{p} {( {σ}_{p}^{k} )}^{2} .$$

The model prediction for the integrated intensity over the image for titer step k would then be simply:

$${H}^{k} = N ( 1 - {e}^{- {k}_{o n} {t}_{s} {α}_{k}} ) + M {α}_{k} + Q$$

with the least-squares sum now defined explicitly as:

$$C ( {k}_{o n} ; N , M , Q ) = {\sum }_{k} {\frac{1}{{( {σ}^{k} )}^{2}} [ ( N ( 1 - {e}^{- {k}_{o n} t {A}_{k}} ) + M {α}_{k} + Q ) - {I}^{k} ]}^{2}$$

Minimization of C allows determination of the optimal parameters corresponding to kₒₙ, N, M, and optionally Q. As there is only S=1 super-pixel per image, the total number of data points is simply equal to the total number of images, Δ=K×S=K. In order to uniquely determine the optimal parameters for a model, the number of data points, Δ, must be equal to or greater than the number of model parameters, Ω, i.e. Δ≥Ω. In the case of no significant background or offset, only the Ω=2 model parameters kₒₙ and N remain, requiring that Δ=K≥Ω=2 images or, simply, K≥2 images. If a significant linear background (slope M) might also be present, then Ω=3 with therefore K≥3 images required. If both a linear background and offset are present, then Ω=4 with then K≥4 images required.

In the case of a global analysis based on the fitting of either single pixel or super-pixel intensities, fewer images are generally required to constrain the model now described by both global and local parameters. A global analysis can also provide a better estimate of the global parameter(s) (in the above case, the kₒₙ). For a global analysis, 22 corresponds to the sum of the total global model parameters, G, and total local model parameters, L, required to fully define the model for each super-pixel, S. The total model parameters are then: Ω=L×S+G, with the total data points, Δ=K×S. The above-stated requirement of more data points than model parameters of Δ≥12, then becomes more explicitly:

$$K \times S \geq L \times S + G$$

$$or , simply :$$

$$K \geq L + G / S$$

As S is typically much larger than G, G/S<1 and the minimum number of required images is simply one more than the number of local parameters, or K=L+1. The local parameters are typically just the normalization factors for the separate model components (e.g. the function describing the signal and the function describing the background), so the number of required images is just one more than the number of separate model components, and therefore independent of the number of global parameters required to define the “shape” of the model component functions. For example, for a global analysis one can easily accommodate a more complicated model for the background than a simple linear model. The above assumes that the individual super-pixels are sufficiently heterogeneous in the contribution of each model component. If the signal to background ratio is always identical, then the above calculation will not hold, but this is extremely unlikely upon consideration of a sufficient number of super-pixels.

An example of the power of global analysis is given in FIG. 2 based on a 2×2 rebinning of the full resolution images into super-pixels. Global analysis returns a value for kₒₙ that agrees very well with the integrated image analysis, showing no real advantage in this particular example for more precise determination of the on-rate constant. However, the more pertinent advantage of global analysis is its ability to optimally infer the fraction of the observed intensity in each super-pixel contributed by signal vs. background. The true signal in each pixel, at least to the fundamental limit set by noise, can then be determined (“Signal” image in FIG. 2), with the background in each pixel equally accessible (“Background” image in FIG. 2). For global analysis, the optimal global and local parameters are determined by minimization of C, with each observed titer image modeled as a simple scaled sum of a trial “Signal” and “Background” images for each step in the minimization until the (typically) unique minimum is obtained. In the above treatment, we have assumed no photobleaching (of the immunofluorescence or sample autofluorescence) takes place during the acquisition of the images for each titer. If photobleaching due to image acquisition is significant, calibration of photobleaching across the image should be performed with the predicted model intensities corrected accordingly.

### Examples

More specifically, FIG. 2 shows an example of the titration process applied to a few micron thick slice of human tonsil tissue fixed with paraformaldehyde. First row: Nuclear stainings (DA PI) of the tissue slice following incubation with different titrations (additive concentration indicated above each image) of the dye-labeled molecule binder. Second row: Immunofluorescence images of the dye-labeled molecule binder (same contrast for all images). Third row: Immunofluorescence images of the dye-labeled molecule binder (independent contrast for each image). Fourth row: Extracted “Signal” and “Background” images from the global analysis of the immunofluorescence image series. Lower left: Fitting of a specific binding model (triangles) to the integrated intensity (circles) of the immunofluorescence images for each additive concentration. Lower right: Fitting of a model (triangles) consisting of the sum of a model for specific binding (diamonds) plus linear background (squares) to the integrated intensities (circles) of the images for each titer. The optimal on-rate constant obtained by global analysis is also displayed for comparison (“Single pixel fit”).

The tissue shown in FIG. 2 was prepared from a fresh frozen tissue block by cryo-sectioning to 8 μm thickness and placement on a coverslip glass onto which a plastic frame containing well structures was mounted. The thin section was then fixed in the well with paraformaldehyde (4% PFA solution), washed with PBS, stained with DA PI, washed with PBS, and then left in buffer. The plate was then mounted in the sample holder of the MA CSima Imaging Platform (Kinkhabwala et al., Sci Rep 12, 1911, 2022).

The titration method was then applied on the M A CSima Imaging Platform in the following manner. First, regions of interest were manually selected from an overview image obtained at low magnification of the DA PI staining. The instrument first photobleached each chosen ROI for 10 min with high-powered LED light. Image acquisition of each ROI was then performed by moving the stage to the saved lateral position (x, y), determining the optimal focus (z) based on imaging of DAPI, acquiring the in-focus DA PI image, and then acquiring an image in the FITC channel of residual autofluorescence. Next, 0.625 μg/mL of FITC-labeled anti-CKHMW (FabREAL 645, Miltenyi Biotec B.V. & Co. KG) was applied to the sample for 10 min. The sample was washed and then in-focus images were obtained of all ROIs in the DAPI and FITC channels, with exposures and excitation intensities chosen to avoid significant photobleaching. This was repeated for each subsequent titration, with the additive concentration labeling the separate images at the top of FIG. 2. In the top row of FIG. 2, the DAPI images are shown of a single ROI for each titration step. The immunofluorescence images from the FITC channel are displayed immediately below in the second row, with all images at the same contrast. The increase in intensity is clear upon incubation of the sample with more and more antibody. In the third row, the separate immunofluorescence images are redisplayed at distinct contrast levels to reveal the pattern of staining. If no background binding occurs, then the pattern of staining should be independent of the exact level of incubation with the antibody. However, perusal of the image series across the third row shows a clear change in the pattern over a gradual increase in titration from left to right.

The image series was then analyzed in the following manner. A simple model comprised of a specific binding model characterized solely by the kₒₙ (1.096×10⁴ M⁻¹ s⁻¹) was used to fit the integrated intensities of the images in the series. While a respectable fit was obtained, further analysis showed that this fit should not be trusted. A more complicated model comprised of an additional linear background term was then used to fit the integrated intensities, for which we obtained a slightly better fit of the intensities but for a very different value of the kₒₙ of 2.457×10⁴ M⁻¹ s⁻¹. Based only on the quality of the fits to the integrated intensities of the images, it is difficult to decide which fit is actually more valid.

A global analysis, however, was then carried out on the image series that demonstrated the superiority of the more complicated model having an additional background contribution. In a global analysis, each individual partition of the data (in this case, each pixel or super-pixel) is considered separately. Global analysis is particularly suitable when the different model components contribute heterogeneously to each data partition, with more reliable estimation possible with global analysis as compared to pooling of the data into a single dataset (the above integrated intensity) and then fitting. Global analysis was first empirically shown to be more accurate for the fitting of fluorescence lifetimes across multiple decay curves obtained from single cuvettes vs. fitting to a single “pooled” decay curve obtained by directly summing over all of the cuvette data (Knutson et al., Biochem 22, 6054, 1983). Global analysis was later extended to imaging data for single pixel fitting of the relative contributions of multiple lifetime components for images obtained with fluorescence lifetime imaging microscopy (Verveer et al., Biophysical Journal 78, 2127, 2004). In a global analysis, minimization is performed jointly with respect to a set of global parameters (e.g., the single global parameter of kₒₙ) and the local parameters relevant for each partition (in our case, the single pixel parameters corresponding to the specific binding normalization, Nₚ, and the background slope, Mₚ). No additional offset parameter (Qₚ in the above mathematical treatment) was required for the fitting of the particular image series displayed in FIG. 2. Global analysis can often yield an even more reliable estimate of the global parameter; however, in this case the global fit based on single pixel fitting yielded a very similar value for the kₒₙ of 2.456×10⁴ M⁻¹ s⁻¹ compared to the integrated intensity value above of 2.457×10⁴ M⁻¹ s⁻¹. The additional advantage of a global analysis, however, is the ability to extract the separate contributions from the model components in each aligned pixel of the image series. In this case, it allows extraction of a “Signal” image containing only the contribution from specific binding interactions in each pixel and a “Background” image. Addition of the “Signal” and “Background” images, using appropriate scale factors also obtained by the global analysis, would then be expected to reproduce each individual titration image. Note the very different staining patterns displayed by the “Signal” and “Background” images in FIG. 2. It is clear for this tissue slice that, for example, the brighter stained cells on the left side of the tissue slice arise solely from non-specific background binding.

1. A method for determining the on-rate constant for specific binding of a conjugate comprising a fluorescent detection moiety and an antigen binding moiety applied to a fixed biological sample expressing the corresponding antigen characterized by the steps
a. measuring the emission radiation of the fixed biological sample as an image formed on a camera before providing the conjugate
b. providing the conjugate to the fixed biological sample subsequently in at least two different concentrations and for a specified time interval
c. detecting the emission radiation for each concentration as an image formed on a camera
d. aligning the images to each other
e. fitting a function that accounts for the amount of specific binding and background binding for each concentration to the emission radiation within each aligned pixel over the separate images
f. obtaining from step e) the on-rate constant that specifies the specific binding function.

2. The method according to claim 1 characterized in that the contribution to the emission radiation of specific binding and background binding in each aligned pixel of the image of the fixed biological sample is determined.

3. The method according to claim 1 characterized by
h. creating an image of specific binding by assigning the emission radiation contributed by specific binding to each aligned pixel
i. creating an image of background binding by assigning the emission radiation contributed by background binding to each aligned pixel.

4. The method according to claim 1 further characterized in that before step d), the following steps are performed
j. waiting a specific time interval
k. detecting the emission radiation as an image formed on a camera

5. The method according to claim 1 further characterized that in step f)
l. from step e) the off-rate constant that specifies the specific binding function is performed

6. The method according to claim 1 further characterized by fitting the function in step e) using a global analysis.

7. The method according to claim 1 further characterized by fitting the function in step e) in two steps
m. in a first step, fitting to the integrated image intensities to determine the specific binding function
n. in a second step, fitting for the amount of specific binding and background binding for each concentration to the emission radiation within each aligned pixel over the separate images
